(19) United States
(12) Patent Application Publication
(10) Pub. No.: US 2009/0229617 A1
(43) Pub. Date: Sep. 17, 2009

(54) Interactive apparatus

(76) Inventor: Simon Allan Bowman, Devon (GB)

Correspondence Address: Melvin I. Stoltz, Esq., 51 Cherry Street, Milford, CT, 06460, US

(21) Appl. No.: 12/077,352

(22) Filed: Mar. 17, 2008

(51) Int. Cl. A61G 15/00; A61F 5/00

(52) U.S. Cl. 128/845; 600/38

(57) ABSTRACT

A primarily movement-based, biomechanically advanced interactive apparatus, that can be operated via simultaneous hand and feet action to encourage participation of all major joints/muscles of the body and which is designed to work with the body in motion.

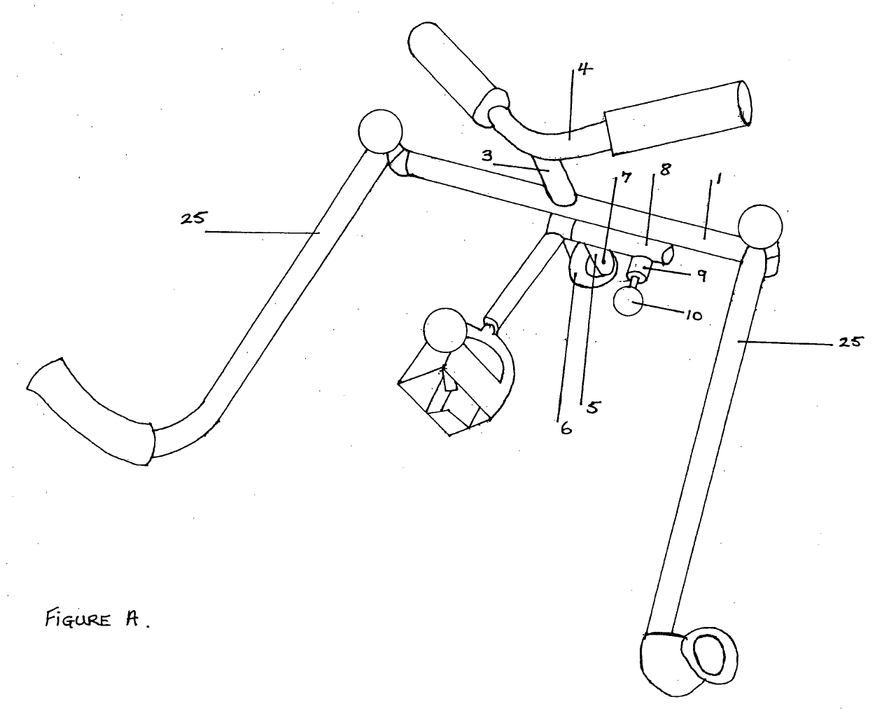

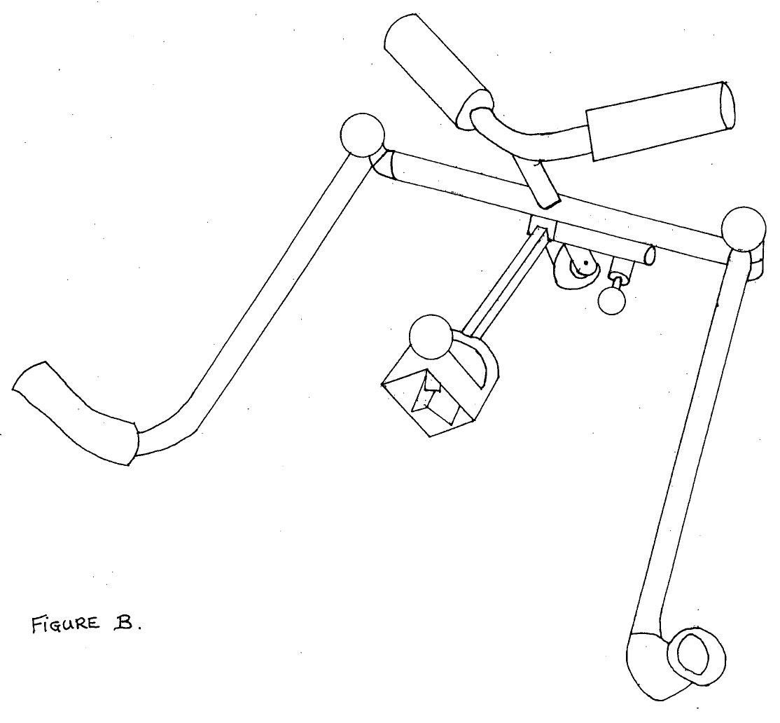

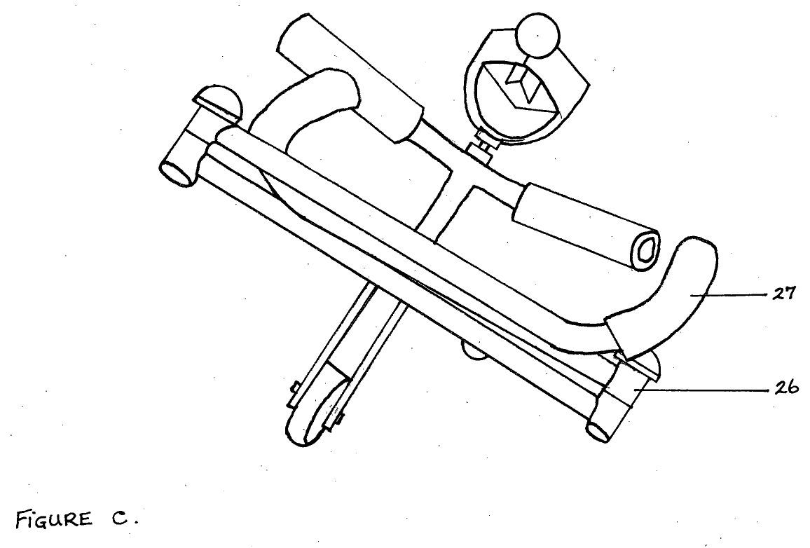

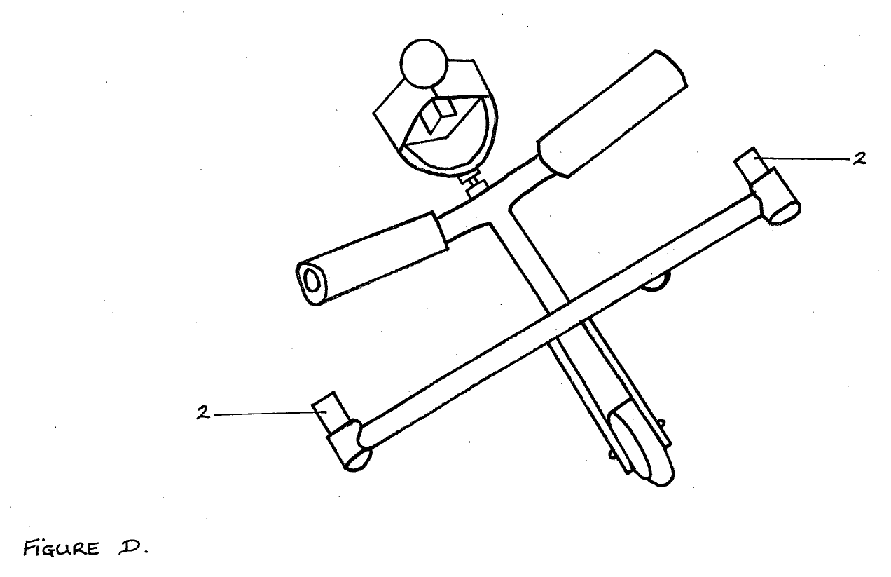

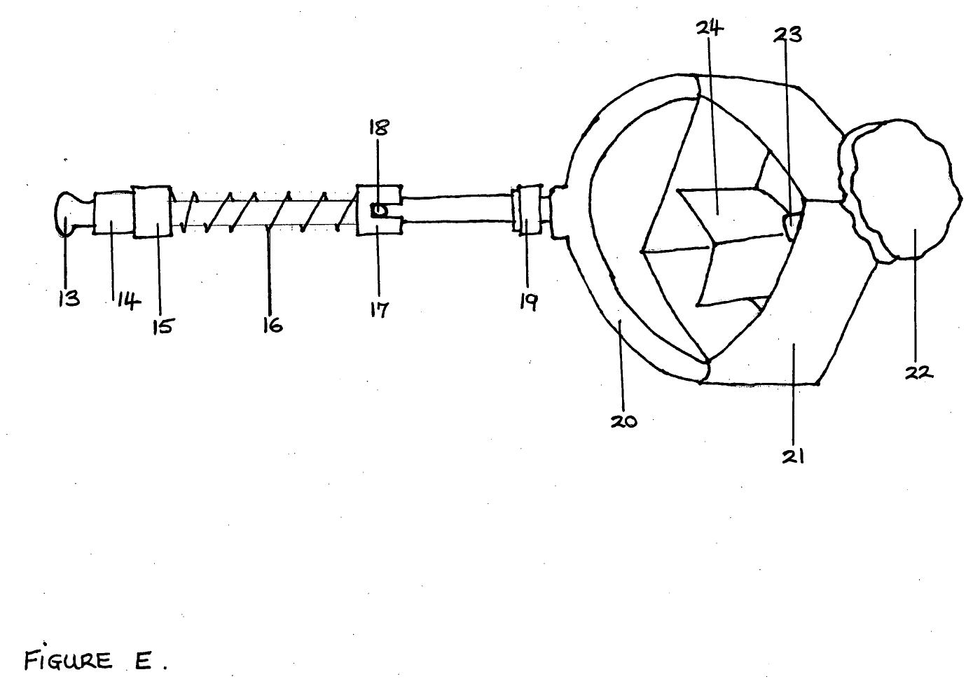

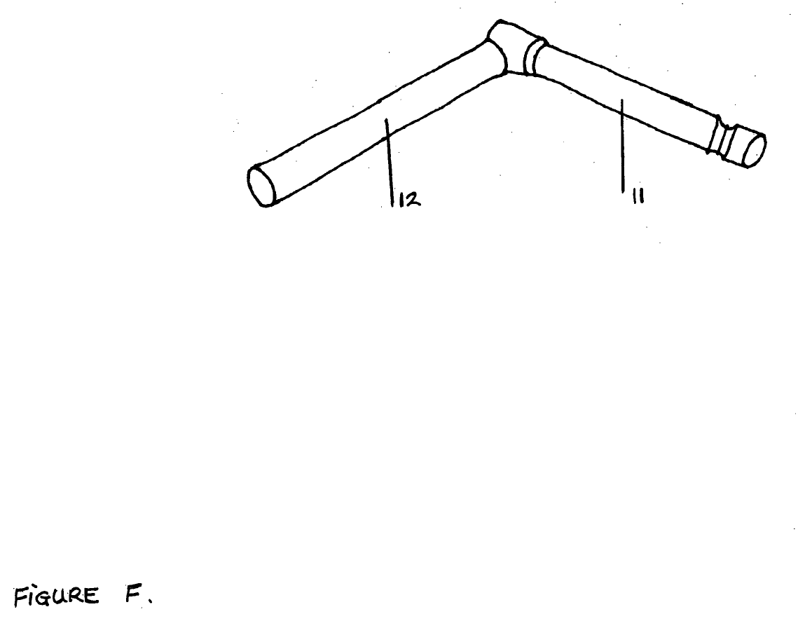

### FIELD OF INVENTION

This invention relates to a new form of interactive apparatus that is designed as a carrier for a wide range of prosthesis of an adult nature and which can provide user-controlled multi-plane movement patterns for the same.

### BACKGROUND TO THE INVENTION

There have been many attempts at creating machinery to simulate that of the male performer during the act of intercourse.

Usually motor-driven and operating in a fixed plane, these often, crude/cumbersome/expensive, mechanical devices are bio-mechanically insufficient, in that they fail to work in harmony with or respond instantaneously to the individual needs of the user.

In view of the low-tech nature of the apparatus currently on offer within this field, there is a clear need for sophisticated apparatus, which is compact, relatively inexpensive and which, due to its bio-mechanical design, offers fully-controlled, user-defined movement patterns to work in complete harmony with the operator.

### SUMMARY OF THE INVENTION

According to the present invention there is provided an interactive apparatus which includes a self-standing base or support structure that includes means for the attachment of two levers, which can remain with the support structure or be removed from the support structure for storage purposes, the levers each having positioned at their superior distal ends hand grips for gripping thereof, and a fixing point on the base or support structure for a centrally positioned fixed or removable coupling, the coupling serving for the mounting of a prosthesis.

The arrangement is preferably such that movement of the levers results in movement of the coupling and hence of the prosthesis.

Means may be provided on the base or support structure for the attachment of a centrally-mounted, fixed position or fully height-adjustable wheel or a ball within a socket or a slide assembly, such as the use of a smooth plastic disk.

The apparatus can also be provided with a base or attachment that replaces the above described assemblies and which reduces, restricts or prevents movement of the apparatus relative to the surface with which it is in contact.

Means may also be provided for the mounting of an angled footrest on the base or support structure.

A hardened or plastic bush may be set into the proximal end of each training lever. The handgrips at the distal ends of the levers may be straight, angled or curved.

### BRIEF DESCRIPTION OF THE DRAWINGS

FIG A is a perspective view of the apparatus fitted with an angle adjustable and rotatable clamp,

FIG B shows the apparatus fitted with a non-rotatable angle adjustable clamp,

FIG C shows the apparatus folded for storage,

FIG D shows the main frame of the apparatus minus levers,

FIG E is an exposed view of the indexing mechanism, and

FIG F shows the indexing mechanism housing tube.

### DESCRIPTION OF THE PREFERRED EMBODIMENT

The principal components of the apparatus are set out below:—

### The Main Support

This includes a support structure consisting of a length of tube 1, attached to the tube can be found two (one at each distal end) vertically extending spigots 2. Extending vertically and centrally positioned on this first section of tube 1 can be found a second length of tube 3. Mounted on the superior aspect of this vertical section can be found an angled or curved length of tube 4 with this section serving as a footrest (for both feet).

Positioned inferiorly to the afore-described vertical prominence 3 and attached to the main support section 1 can be found two equally spaced metal extensions 5. The extensions 5 serve for the positioning between them of a single wheel 6 which is held in place by a single bolt 7 that passes through each drilled extension 5 and the centre of the wheel 6. The extensions 5 can be drilled along their length, so that height adjustment can be made by the inferior or superior positioning of the wheel 6 upon the drilled extensions.

A short length of tube 8 is positioned on the main frame section 1 and is laterally drilled to accept a threaded boss 9 together with a threaded plunger/hand wheel assembly 10. This arrangement serves as a removable fixing point for the coupling unit described next.

### The Coupling Unit.

Referring to the coupling unit, it must be noted that several products can be provided utilising the principles described herein, with the following examples representing just a few.

Product one is provided with an angle-adjustable, non-rotatable coupling unit.
Product two is provided with angle adjustment together with an indexed rotatable coupling unit.
Product three could include a motorised (thruster) unit, that is removably fixed to the main frame, thus complementing product evolution.

A coupling unit is now described that allows for angled adjustment and/or indexed rotation of the prosthesis.

A length of bar 11 is provided that has nearing its distal end a groove that is formed about its full circumference. A hand wheel and threaded shank assembly 10 serves to secure the bar 11 within the frame-mounted tubular housing 8, allowing it to rotate or be fixed, depending on the amount of torque exerted upon the hand wheel.

Welded to the proximal face of the bar 11 can be found a short length of tube 12. The tube 12 serves to house an indexing mechanism that allows for the accurate positioning of the clamp assembly. The indexing mechanism consists of a short length of slide bar 13 that has an expanded portion at one end. Having been positioned over the bar to butt up against the expanded portion, there can be found a ring collar 14. Next to the ring collar 14 can be found a rotatable spring-retaining bush 15 and, communicating with this bush 15, a compression spring 16 spans the first spring-retaining bush 15 and a further spring-retaining bush 17 that has on the opposite end to its spring-communicating side a milled slot. Lying within the slot and passing through a hole on the slide bar 13 can be found a hardened steel pin 18.

Forward of the said pin 18 and also positioned upon the slide bar 13 can be found a bush 19 that acts as a nose and slide bar guide cone when the mechanism is fully assembled. Welded to the opposite end of the expanded portion of the slide bar 13 can be found a curved section 20, whose distal ends communicate centrally with an upended rectangular section 21.

A hole (not shown) is drilled in the uppermost corner of the rectangular section 21 and an internally threaded boss (not shown) is secured to the outside of the corner. A threaded shank passes through the threaded boss (not shown). There is a knob 22 at the outer end of the shank and the inner end of the shank has engagement in a bush 23 that is in turn welded to a short section of angle iron 24. The section 24 provides an abutment face that cooperates with the internal face of the corner of section 21 to provide two opposing abutment faces.

In use, the shank (not shown) is moved into a retracted position and the prosthesis (not shown) is placed in the space between the angle section 24 and the corner of section 21. The said shank is then rotated to move the angle section 24 towards the corner of the section 21 and movement is continued until a firm grip is exerted on the prosthesis. Various shaped bushes can be used in place of section 24 to accommodate different prosthesis together with differing clamp arrangements to suit different applications.

To assemble the above unit, the slide bar 13 and associated spring mechanism is placed within the tube section 12 and the tube section 12 is spot-welded to section 17 of the indexing mechanism and the nose cone 19 slid to communicate partly within the tube section 12. The bar 11 is then positioned within the tube section 8 and held in place by the threaded shank/knob assembly 10.

### Angle Adjustment

To adjust the coupling up or down, the knob 10 is turned anti-clockwise, thus allowing for rotation of the bar 11 within the housing 8. The required angle is chosen, then the knob 10 is turned clockwise thus bearing down upon the bar 11 and fixing the coupling in position.

### Indexed Rotation

The indexed rotation facility is specifically designed for use with the Rabbit vibrator or similar design, where the user may wish to utilise the clitoral stimulator before inverting/righting the vibrator to facilitate intercourse.

Indexed rotation is achieved by pulling the clamp 21 towards the user thus disengaging the pin 18 from the slot on section 17, rotating it one half turn and then allowing the pin 18 to engage the slot via spring tension 16 to reposition the clamp in an opposite orientation.

The apparatus may be provided without an angle adjustment or rotation facility:—

Clamp minus rotation is achieved by welding a bar section between the rectangular section and the angle adjusting bar.

Clamp minus angle adjustment is achieved by welding a bar between the rectangular section and the main frame.

If rotation without angle adjustment is required, then the indexing mechanism housing tube is welded directly to the main frame.

Rotation without an indexing facility can be provided in a number of ways to include a simple collar, rod and grip nut assembly.

It may be noted that the apparatus can serve as a carrier or to provide movement patterns for a wide variety of motorised/non-motorised forms of prosthesis of an adult nature.

### The Levers

Each lever 25 comprises a length of metal tube with a bush 26 vertically set into or fixed to its most distal aspect. The proximal aspect of each lever 27 is curved and acts as a handgrip. Each handgrip is angled slightly outwards to maximise bio-mechanical and ergonomic efficiency.

In addition to providing movement of the support structure and thus movement for the prosthesis (not shown) via the levers, one or both handgrips can be fitted with an actuator switch. The actuator switch serves to provide remote control for certain types of prosthesis and can be activated for example via a button or by throttle control functioning of one or both handgrips. For example, the right hand lever mounted control could operate the head of the vibrator and the left hand mounted control could operate a clamp mounted-multi positional clitoral stimulator and vice versa.

The apparatus provides ample space for the positioning of a larger or more efficient (to that of a hand held prosthesis) power or battery pack.

Each lever can be telescopic in construction to allow for differing body proportions and to facilitate improved breakdown for storage. The levers are held in position on each bush by threaded locking means that can be tightened down completely to prevent rotational movement of one or both levers.

### Mechanical Movement Patterns & Bio-Mechanics

The present invention is primarily concerned with the provision of movement/control of the prosthesis and the obtaining of bio-mechanical compatibility of the apparatus with the user, as described below.

### Mechanical Movement

The prosthesis is centrally mounted upon the main support structure of the apparatus and secured in place by one of the previously described coupling units. The levers are pivotally mounted at their distal ends to the distal aspects of the main support frame so that, when activated (pushed/pulled, etc) they provide movement for the coupling unit and thus for the prosthesis.

For example, if both levers are pushed or pulled together, in linear fashion, then anterior/posterior movement of the prosthesis will occur. If, for example one of the handgrips were to be pulled whilst the other was moved forwards then pivotal movement would occur at the wheel and the prosthesis would incur lateral movement. On lifting or lowering the handles, corresponding upward and downward movements of the prosthesis will be obtained.

The apparatus shown in the Figures has been primarily designed to be operated with both hands and feet working simultaneously, so that maximum control of the apparatus and prosthesis can be achieved by the operator during use.

In use, the apparatus works with the operator under nominal load and, importantly, with all associated joints being allowed complete freedom of movement therefore assuring user safety.

It will be appreciated from the above that the apparatus is so designed that multiple movement patterns in respect of movement of the prosthesis can be engaged, with changes to these occurring instantaneously and under full control of the operator.

It is also to be appreciated that, although the accompanying Figures show an apparatus that is formed by welding together of metallic components, a similar construction offering the same movement patterns can be produced by the utilisation of the plastic injection moulding process.

It may be noted that, in order to enhance sensory enjoyment of the apparatus for the user, integral or external vibration pads/devices may be fitted to both hand and feet contact points, i.e. to the handgrips and footrests. The vibratory facilities may include various vibration/pulse settings and operate together or randomly throughout the contact points.

1. An interactive apparatus which includes a self-standing base or support structure that includes means for the attachment of two levers, which can remain with the support structure or be removed from the support structure for storage purposes, the levers each having positioned at their superior distal ends hand grips for gripping thereof, and a fixing point on the base or support structure for a centrally positioned fixed or removable coupling, the coupling serving for the mounting of a prosthesis.

2. Apparatus according to claim 1, in which an elevated, ergonomically configured foot rest is centrally positioned upon the frame or support structure, and two distally positioned spigots (upon the frame) provide pivot points for the two levers.

3. Apparatus according to claim 2, in which the levers are so arranged that, when the levers are pushed/pulled, via the handgrips, in an alternating linear fashion together with associated independent foot action, serve to provide lateral (side to side) movement for the coupling.

4. Apparatus as claimed in claim 1, in which means are provided on the frame or support structure for attachment of a centrally-mounted, fixed position or fully height adjustable wheel, to provide support for the weight of the operator's lower limbs.

5. An interactive apparatus as claimed in claim 4, in which a multi-positional clitoral stimulator is attached to or forms an integral part of the coupling.

6. Apparatus as claimed in claim 1, wherein vibratory pads are placed at both foot and hand contact points of the frame or support structure.
